(19) United States
(12) Patent Application Publication
(10) Pub. No.: US 2013/0236904 A1
(43) Pub. Date: Sep. 12, 2013

(54) METHOD FOR SCREENING FOR MATERIALS FOR PROMOTING THE DIFFERENTIATION OF SKIN CELLS

(71) Applicants: Hyun Choi, Yongin-si (KR); Yeun Kyung Kim, Seoul (KR); Seung Ha Yang, Seoul (KR); Tae Ryong Lee, Yongin-si (KR); Min Soo Noh, Seoul (KR); Dong Wook Shin, Seoul (KR)

(72) Inventors: Hyun Choi, Yongin-si (KR); Yeun Kyung Kim, Seoul (KR); Seung Ha Yang, Seoul (KR); Tae Ryong Lee, Yongin-si (KR); Min Soo Noh, Seoul (KR); Dong Wook Shin, Seoul (KR)

(73) Assignee: AMOREPACIFIC CORPORATION, Seoul (KR)

(21) Appl. No.: 13/988,270

(22) PCT Filed: Nov. 18, 2011
(86) PCT No.: PCT/KR11/08838

§ 371 Date: May 17, 2013

(30) Foreign Application Priority Data
Nov. 19, 2010 (KR) 10-2010-0115853

(51) Int. Cl. C12Q 1/68

(52) U.S. Cl. CPC C12Q 1/6881
USPC 435/6.12; 435/6.13

(57) ABSTRACT

The present invention relates to a method for screening for materials for promoting the differentiation of skin cells, comprising: a step of treating skin cells with a test material; and a step of checking the relative degree of the expression of the DUOX 1 gene in the skin cells treated with the test material in the previous step. The present invention also relates to a kit for screening for materials for promoting the differentiation of skin cells, comprising a device for checking the relative degree of expression of the DUOX 1 gene in skin cells. The present invention also relates to a composition which contains, as an active ingredient, a material for increasing the expression of the DUOX 1 gene to promote the differentiation of skin cells, moisturize the skin, improve skin barrier function, or alleviate or treat symptoms of atopic dermatitis.

### TECHNICAL FIELD

This disclosure relates to a method for screening materials for promoting differentiation of skin cells and a kit therefor.

### BACKGROUND ART

The epidermis is the outermost layer of the skin, and the stratum corneum is the outermost layer of the epidermis. When the stratum corneum, composed of keratinocytes, is in normal state, the epidermis acts as the body's major barrier against various stimulations and prevents the emission of moisture from the body. The keratinocytes proliferate in the basal layer, the innermost skin layer, and differentiate gradually as they pass through the spinous layer and the granular layer. Through this keratinization process, the keratinocytes produce natural moisturizing factors (NMFs) and lipids (ceramides, cholesterols or fatty acids), and form the stratum corneum. Through this normal formation of the stratum corneum, the skin barrier function may work right.

### DISCLOSURE

### Technical Problem

This disclosure is directed to providing a method for screening materials for promoting differentiation of skin cells and a kit for screening materials for promoting differentiation of skin cells. Further, the disclosure is directed to providing a composition for promoting differentiation of skin cells, moisturizing skin, strengthening skin barrier function, or reducing or treating symptoms of atopy.

### Technical Solution

In one aspect, there is provided a method for screening materials for promoting differentiation of skin cells including: treating skin cells with a test material; and verifying a relative expression level of dual oxidase 1 (DUOX 1) gene in the skin cells treated with the test material in the previous step.

In another aspect, there is provided a kit for screening materials for promoting differentiation of skin cells including a device, which may verify a relative expression level of DUOX 1 gene in skin cells.

In further another aspect, there is provided a composition for promoting differentiation of skin cells, moisturizing skin, strengthening skin barrier function, or reducing or treating symptoms of atopy, which includes materials increasing expression of DUOX 1 gene as an active material.

### Advantageous Effects

According to the method for screening materials for promoting the differentiation of the skin cells disclosed herein, the method may screen the materials for promoting the differentiation of the skin cells conveniently, rapidly and efficiently by verifying the relative expression level of DUOX 1 gene after treating the skin cells with the test material.

### DESCRIPTION OF DRAWINGS

FIG. 1 is a graph showing expression level of DUOX 1 gene according to calcium concentration in normal human epidermal keratinocytes.

FIG. 2 is a graph showing expression level of DUOX 1 or DUOX 2 when treating DUOX 1 or DUOX 2 siRNA to skin epidermal keratinocytes, compared with the case not treated with them.

FIGS. 3 and 4 are graphs showing expression level of filaggrin and loricrin (FIG. 3), and keratin 1 and keratin 10 (FIG. 4) when treating DUOX 1 or DUOX 2 siRNA to skin epidermal keratinocytes, compared with the case not treated with them.

FIGS. 5 and 6 are graphs showing expression level of DUOX 1 and filaggrin (FIG. 5), and keratin 1 and keratin 10 (FIG. 6) when treating an extract of saururus or melia to skin epidermal keratinocytes, compared with the case not treated with them.

### BEST MODE

Dual oxidase 1 is also called Duox 1 or ThOX 1 (Thyroid oxidase), and encoded by DUOX 1 gene. This was first found in the thyroid gland. There are two isotypes of hDUOX 1 and hDUOX 2 in human, and the hDUOX 1 is largely expressed in airway epithelial cells, and hDUOX 2 is largely expressed in the salivary gland and the gastrointestinal tract. The Duox 1 belongs to NADPH oxidase (NOX) having total 7 isotypes (Nox 1, Nox 2, Nox 3, Nox 4, Nox 5, Duox 1 and Duox 2). Of them, the NOX has been much studied as a protein producing reactive oxygen species (ROS) in bacterial phagocytosis of phagocytes, but recently, various isotypes are also found in cellular tissues other than immune cells and studied. The NOX/DUOX group is known to have relevance to hypertension (NOX 1), immune (NOX 2/DUOX), ear formation (NOX 3), thyroid hormone production (DUOX 1 and 2) and the like through the biological role of producing reactive oxygen.

The stratum corneum constituting the outermost layer of the skin barrier is made up of natural moisturizing factors, lipids and the like. Filaggrin protein is degraded to many kinds of hydrophilic amino acids through a post-transcriptional modification process, and it is known that the resultant amino acid pool forms the natural moisturizing factors (NMFs), and the NMFs help maintain moisture of the stratum corneum. Further, it is also known that mutation of filaggrin gene is an essential genetic risk factor inducing atopic eczema by reducing skin moisturizing factors, damaging skin barrier function, reducing skin defense ability against allergens or microbes and activating T cell groups thereby causing chronic inflammation by auto-immune mechanism. However, methods for treating or preventing thereof are not presented yet.

Hereinafter, the present disclosure will be described in detail.

The inventors verified that DUOX 1 is relatively largely expressed in normal human epidermal keratinocyte of the skin, and expression level thereof is increased during differentiation process. Further, it was confirmed that the expression of DUOX 1 has relevance to the expression of filaggrin, loricrin, keratin 1 and keratin 10 genes involved in the skin cell differentiation, by verifying increase of expression level of markers of various differentiation genes such as loricrin, keratin 1 and keratin 10 together with filaggrin gene when deleting DUOX 1 (siRNA). Based on this, it may be decided that the materials increasing the expression level of DUOX 1 gene in skin cells are materials for promoting the skin cell differentiation. Namely, whether a random material is a material for promoting the skin cell differentiation or not may be easily verified by checking the relative expression level of DUOX 1 gene after treating the random material to the skin cells.

One embodiment of the present disclosure provides a method for screening materials for promoting differentiation of skin cells including: treating the skin cells with a test material; and verifying a relative expression level of dual oxidase 1 (DUOX 1) gene (Genebank Accession No.: NM_{—}017434) in the skin cells treated with the test material in the previous step.

In this specification, the term “skin” implies tissues covering the surface of an animal body, and is the most broadly-defined concept including the scalp and hair as well as the tissues covering the surface of a face or a body.

In one embodiment of the present disclosure, the skin cells include skin epidermal keratinocytes. In another embodiment of the present disclosure, the skin cells include skin epidermal keratinocytes of human. In further another embodiment of the present disclosure, the skin cells include normal skin epidermal keratinocytes of human.

In this specification, the term “relative expression level” may be the expression level when comparing with the expression level of expression of DUOX 1 gene in skin cells not treated with a test material. The expression level includes expression amount and expression quality.

After verifying the relative expression level of DUOX 1 gene according to one embodiment of the present disclosure, deciding materials, which increase the expression level of DUOX 1 gene as materials for promoting the differentiation of skin cells, may be further included. Specifically, when the expression level of DUOX 1 gene in the skin cells treated with the test material is higher than the expression level of DUOX 1 gene in the skin cells not treated with the test material, it may be decided that the treated test material increases the expression level of DUOX 1 gene. On the contrary, when the expression level of DUOX 1 gene in the skin cells treated with the test material is lower than the expression level of DUOX 1 gene in the skin cells not treated with the test material, it may be decided that the treated test material does not increase the expression level of DUOX 1 gene. As seen previously, the treated test material increasing the expression level of DUOX 1 gene may be decided as a material for promoting the skin cell differentiation.

In verifying the relative expression level of DUOX 1 gene in the cells treated with the test material according to one embodiment of the present disclosure, the relative expression level may be verified by using RT-PCR, ELISA or western blot (immuno blot).

In addition to verifying the relative expression level of DUOX 1 gene, the method for screening materials for promoting differentiation of skin cells according to one embodiment of the present disclosure may further include verifying the relative expression level of at least one gene, selected from the group consisting of filaggrin, loricrin, keratin 1 and keratin 10, in the skin cells treated with the test material. Whether the subject test material is a material for promoting differentiation of skin cells or not may be more clearly decided by verifying the relative expression level of markers of the genes involved in the skin cell expression.

After verifying the relative expression level of at least one gene, selected from the group consisting of filaggrin, loricrin, keratin 1 and keratin 10, according to one embodiment of the present disclosure, deciding the materials increasing the expression level of the selected at least one gene as materials for promoting the differentiation of skin cells may be further included. Specifically, when the expression level of the selected at least one gene in the skin cells treated with the test material is higher than the expression level of the selected at least one gene in the skin cells not treated with the test material, it may be decided that the treated test material increases the expression level of the selected at least one gene. As seen previously, the treated test material increasing the expression level of the selected at least one gene may be decided as a material for promoting the skin cell differentiation.

If the skin cell is not properly differentiated, the stratum corneum of the skin may not work right. Accordingly, water retention power of the skin may be reduced, and the skin barrier function may be deteriorated. Further, skin diseases such as atopy are diseases caused because functions of the normal skin stratum corneum is not maintained by various factors, and their major symptoms are skin inflammation and dry skin. Thus, the materials for promoting the skin cell differentiation may exert effects of moisturizing skin, strengthening the skin barrier function, and reducing or treating symptoms of atopy. Namely, materials for moisturizing skin, strengthening the skin barrier function, and reducing or treating symptoms of atopy may be screened by using the method for screening materials for promoting the differentiation of the skin cells according to one embodiment of the present disclosure.

One embodiment of the present disclosure provides a kit for screening materials for promoting the differentiation of the skin cells including a device verifying the relative expression level of DUOX 1 gene in the skin cells. The materials for promoting the differentiation of the skin cells may be conveniently, rapidly and efficiently screened by verifying the relative expression level of DUOX 1 gene in the skin cells by using the screening kit.

As seen previously, the materials increasing the expression of DUOX 1 gene exert effects of promoting the skin cell differentiation, moisturizing skin, strengthening the skin barrier function, and reducing or treating symptoms of atopy. Accordingly, one embodiment of the present disclosure provides a composition for promoting the differentiation of the skin cells, a composition for moisturizing skin, a composition for strengthening the skin barrier function and a composition for reducing or treating symptoms of atopy, which include materials increasing expression of DUOX 1 gene as an active material. Another embodiment of the present disclosure provides a composition for promoting the differentiation of the skin cells, a composition for moisturizing skin, a composition for strengthening the skin barrier function and a composition for reducing or treating symptoms of atopy, which include materials increasing the expression of the screened DUOX 1 gene as an active material.

In one embodiment, the composition may be a cosmetic composition. In another embodiment, the composition may be a pharmaceutical composition. In further another embodiment, the composition may be a health food composition.

The examples will now be described. The following examples are for illustrative purposes only and not intended to limit the scope of the present disclosure.

### Example 1

### Evaluation of Change in Expression of DUOX 1 Gene by Calcium Concentration

Generally, it is known that skin cell differentiation is promoted when intracellular calcium concentration is high. This experiment is to evaluate expression level of DUOX 1 gene after changing degree of skin cell differentiation by treating calcium to cells with different concentration.

Normal human epidermal keratinocytes (human epidermal neonatal keratinocyte cells) are purchased from Lonza, Inc. (Walkersville, Md., USA) and subcultured, and then incubated in a CO₂ incubator under a condition of 37° C. and 5% CO₂. Cell culture is prepared according to the instructions of Lonza, Inc. (Walkersville, Md., USA). KGM-2 bullet kit [bovine pituitary extract (BPE), 2 ml), human epidermal growth factor (hEGF, 0.5 ml), insulin (0.5 ml), hydrocortisone (0.5 ml), transferrin (0.5 ml), epinephrine (0.5 ml), gentamycin sulfate+amphofericin B (GA-1000, 0.5 ml)] are added to KBM-2 (Clonetics CC-3103) medium (500 ml).

120 μM and 1.2 mM Calcium are added to the culture medium of human epidermal keratinocytes, respectively, and the cells are incubated for 24 hours. Each of them is used as a low calcium group (control group) and a high calcium group (test group). 24 hours after the interleukin treatment, the cells are washed twice with 10 ml of phosphate buffered saline (PBS) and intracellular total RNA is isolated from the cells using Trizol reagent (Invitrogen, Carlsbad, Calif., USA). The isolated RNA is purified once more using an RNA kit (Qiagen RNeasy kit, Qiagen, Valencia, Calif.) and RNA quality is verified using Agilent 2100 BioAnalyzer (Agilent Technologies, Santa Clara, Calif., USA). cDNA is synthesized from the isolated RNA using a reverse transcription kit (Superscript Reverse Transcriptase (RT) II kit, Invitrogen, Carlsbad, Calif.), and expression of various NOX genes is quantitatively analyzed by means of real time-reverse transcription polymerase chain reaction (Q-RT-PCR). Change in the expression pattern of NOX 1 (Hs00246589_m1), NOX 2 (Hs00166163_m1), NOX 3 (Hs00210462_m1), NOX 4 (Hs00276431_m1), NOX 5 (Hs00225846_m1), DUOX 1 (Hs00213694_m1) and DUOX 2 (Hs00204187_m1) is evaluated using TaqMan° gene expression assay kit (Applied Biosystems, Foster City, Calif.). The result is shown in FIG. 1.

As shown in FIG. 1, in the normal human epidermal keratinocyte, the expression level of DUOX 1 gene is basically high, and the expression of DUOX 1 gene is more remarkably increased when the skin cell differentiation is increased by using calcium. Namely, it may be confirmed that the DUOX 1 gene has relevance to the skin cell differentiation, and specifically, the increase of the expression of DUOX 1 gene has relevance to the skin cell differentiation.

### Example 2

### Evaluation of Change in Expression of Filaggrin Gene and Various Differentiation Markers According to Deletion of DUOX 1

siRNA (SMARTPOOL) which is inhibiting expression of DUOX 1 or DUOX 2 gene is purchased from Dharmacone.

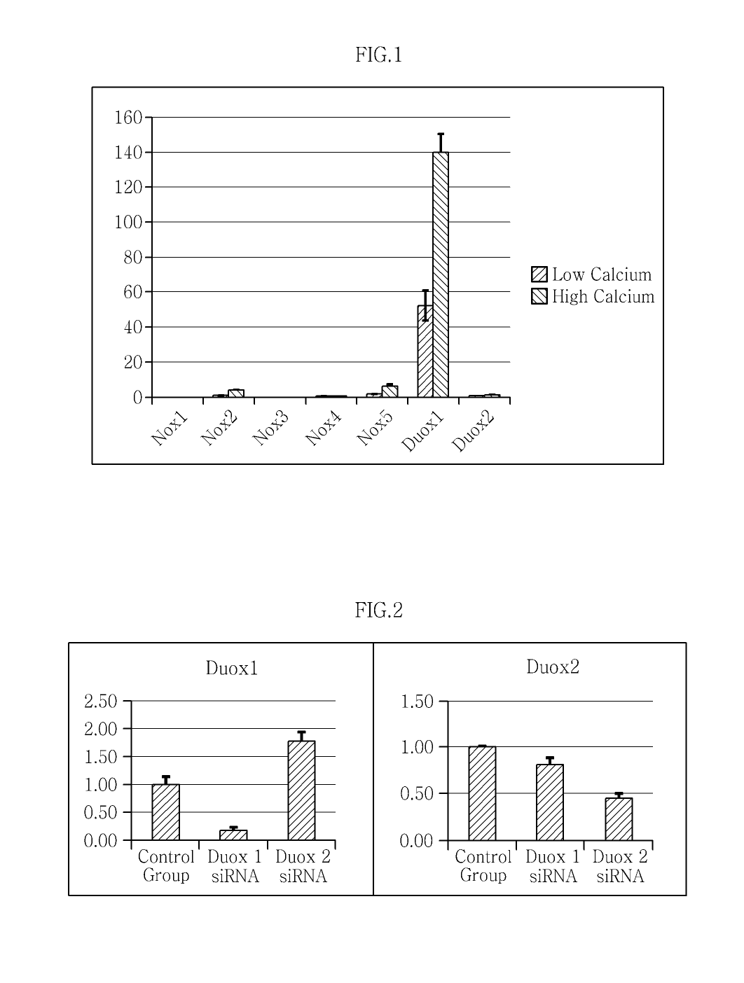

As in Example 1, normal human epidermal keratinocytes are cultured, and then cultured on a 6-well plate, at 2×10⁴ cells/cm². 24 hours later, 50 nM DUOX 1 or DUOX 2 siRNA is transfected using RNAi MAX reagent (Invitrogen), respectively. 6 hours later, cell culture medium is replaced. 24 hours after the transfection, the cells are washed twice with 10 ml of phosphate buffered saline (PBS) and intracellular total RNA is isolated from the cells using Trizol reagent (Invitrogen, Carlsbad, Calif., USA). The isolated RNA is purified once more using an RNA kit (Qiagen RNeasy kit, Qiagen, Valencia, Calif.) and RNA quality is verified using Agilent 2100 BioAnalyzer (Agilent Technologies, Santa Clara, Calif., USA). cDNA is synthesized from the isolated RNA using a reverse transcription kit (Superscript Reverse Transcriptase (RT) II kit, Invitrogen, Carlsbad, Calif.), and quantitatively analyzed by means of real time-reverse transcription polymerase chain reaction (Q-RT-PCR). Change in the expression pattern of DUOX 1 (Hs00213694_m1), and filaggrin (Genebank Accession No.: NM_{—}002016) (Hs00856927_g1), loricrin (Genebank Accession No.: NM_{—}000427) (Hs01894962_s1), keratin 1 (Genebank Accession No.: NM_{—}006121) (Hs00196158_m1), keratin 10 (Genebank Accession No.: NM_{—}000421) (Hs00166289_m1) as marker genes of differentiation of human epidermal keratinocytes is evaluated using TaqMan° gene expression assay kit (Applied Biosystems, Foster City, Calif.). The results are shown in FIGS. 2 to 4.

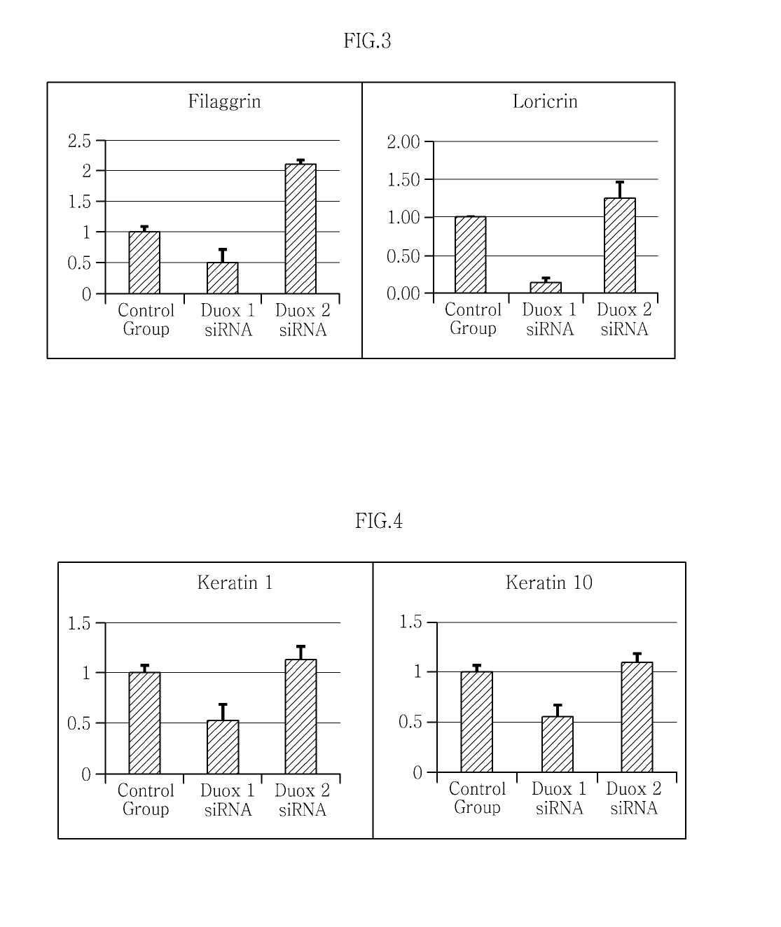

FIG. 2 is a graph showing the expression level of DUOX 1 or DUOX 2 when treated with DUOX 1 or DUOX 2 siRNA, compared with the control group not treated with DUOX 1 or DUOX 2 siRNA. FIG. 3 is a graph showing the expression level of filaggrin and loricrin genes when treated with DUOX 1 or DUOX 2 siRNA, compared with the control group not treated with DUOX 1 or DUOX 2 siRNA, and FIG. 4 is a graph showing the expression level of keratin 1 and keratin 10 genes in the same way.

As shown in the above results, the expression level of DUOX 1 or DUOX 2 gene is reduced when treated with the DUOX 1 or DUOX 2 siRNA. Further, each expression level of filaggrin, loricrin, keratin 1 and keratin 10 genes is also reduced, compared with the case not treated with the DUOX 1 or DUOX 2 siRNA. Namely, it may be confirmed that the expression level of the genes involved in the skin cell differentiation is reduced when deleting DUOX 1.

### Example 3

### Evaluation of Expression Level of Gene Such as DUOX 1 and Filaggrin of Test Material

Expression level of DUOX 1, filaggrin, keratin 1 and keratin 10 is verified by using extracts of saururus and melia known as materials promoting the skin cell differentiation.

Human skin epidermal keratinocytes are purchased from Lonza, Inc. (Walkersville, Md., USA), and cultured in KBM-2 (Clonetics CC-3103) medium in a CO₂ incubator under a condition of 37° C. and 5% CO₂.

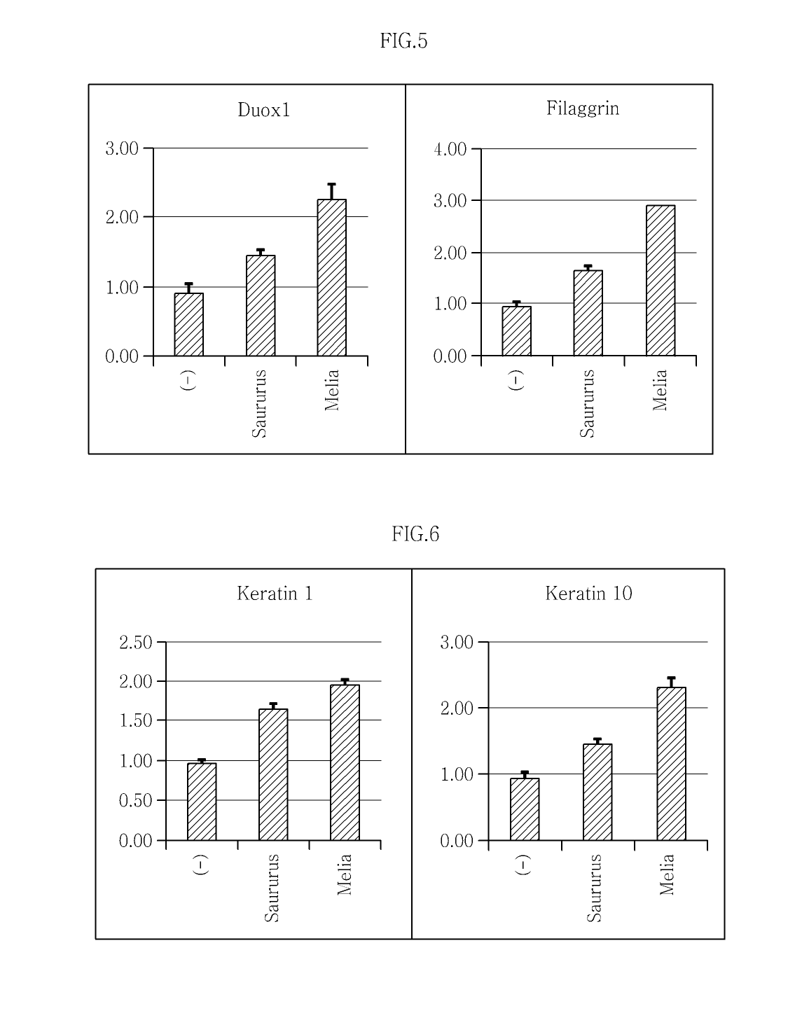

A group of the cultured human epidermal keratinocytes without any treatment (control group) and test groups of the cultured human epidermal keratinocytes with the extract of saururus or melia of 10 μM, respectively are cultured for 24 hours. The extracts of saururus and melia are purchased from Plank Extract Bank in Korea. 24 hours after treating the extract of saururus or melia, the cells are washed twice with 10 ml of phosphate buffered saline (PBS) and intracellular total RNA is isolated from the cells using Trizol reagent (Invitrogen, Carlsbad, Calif., USA). The isolated RNA is purified once more using an RNA kit (Qiagen RNeasy kit, Qiagen, Valencia, Calif.) and RNA quality and concentration are verified using Agilent 2100 BioAnalyzer (Agilent Technologies, Santa Clara, Calif., USA). cDNA is synthesized from the isolated RNA using a reverse transcription kit (Superscript Reverse Transcriptase (RT) II kit, Invitrogen, Carlsbad, Calif.), and change in the gene expression is quantitatively analyzed by means of real time-reverse transcription polymerase chain reaction (Q-RT-PCR). Change in the expression pattern of DUOX 1 (Hs00213694_m1), and filaggrin (Hs00856927_g1), keratin 1 (Hs00196158_m1) and keratin 10 (Hs00166289_m1) as marker genes of differentiation of human epidermal keratinocytes is evaluated using TaqMan° gene expression assay kit (Applied Biosystems, Foster City, Calif.). The results are shown in FIG. 5 and FIG. 6.

FIG. 5 is a graph showing the expression level of Duox 1 and filaggrin genes when treated with the extract of saururus or melia, compared with the control group not treated with them, and FIG. 6 is a graph showing the expression level of keratin 1 and keratin 10 genes in the same way.

As shown in the above results, the extracts of saururus and melia remarkably increase the expression level of DUOX 1 gene, filaggrin, keratin 1 and keratin 10 genes in human epidermal keratinocytes. Namely, it may be confirmed that materials having skin cell differentiation promoting effect, further skin moisturizing, skin barrier function strengthening and atopy symptom reducing or treating effect increase the expression level of the genes.

While the exemplary embodiments have been shown and described, it will be understood by those skilled in the art that various changes in form and details may be made thereto without departing from the spirit and scope of the present disclosure as defined by the appended claims.

1. A method for screening materials for promoting differentiation of skin cells comprising:
treating skin cells with a test material; and
verifying a relative expression level of dual oxidase 1 (DUOX 1) gene (Genebank Accession No.: NM_{—}017434) in the skin cells treated with the test material.

2. The method for screening materials for promoting differentiation of skin cells according to claim 1, which further comprises deciding materials increasing the expression level of DUOX 1 gene as materials for promoting differentiation of skin cells after said verifying a relative expression level of DUOX 1 gene.

3. The method for screening materials for promoting differentiation of skin cells according to claim 1, which further comprises verifying a relative expression level of at least one gene, selected from the group consisting of filaggrin, loricrin, keratin 1 and keratin 10, in the skin cells treated with the test material in addition to said verifying a relative expression level of DUOX 1 gene.

4. The method for screening materials for promoting differentiation of skin cells according to claim 3, which further comprises deciding materials increasing the expression level of at least one selected gene as materials for promoting differentiation of skin cells after said verifying a relative expression level of at least one gene, selected from the group consisting of filaggrin, loricrin, keratin 1 and keratin 10.

5. The method for screening materials for promoting differentiation of skin cells according to claim 1, wherein the skin cells are keratinocytes.

6. The method for screening materials for promoting differentiation of skin cells according to claim 1, which is a method for screening materials for moisturizing skin by promoting differentiation of skin cells.

7. The method for screening materials for promoting differentiation of skin cells according to claim 1, which is a method for screening materials for strengthening skin barrier function by promoting differentiation of skin cells.

8. The method for screening materials for promoting differentiation of skin cells according to claim 1, which is a method for screening materials for reducing or treating symptoms of atopy by promoting differentiation of skin cells.

9. A kit for screening materials for promoting differentiation of skin cells comprising a device verifying a relative expression level of DUOX 1 gene in skin cells.

10. (canceled)

11. (canceled)

12. (canceled)

13. (canceled)

14. A method for improving function of skin stratum corneum comprising administering materials increasing expression of DUOX1 gene to a subject in need thereof, wherein the materials improve function of skin stratum corneum.

15. The method for improving function of skin stratum corneum according to claim 14, wherein the materials increasing expression of DUOX 1 gene promote differentiation of skin cells.

16. The method for improving function of skin stratum corneum according to claim 14, wherein the materials increasing expression of DUOX 1 gene moisturize skin.

17. The method for improving function of skin stratum corneum according to claim 14, wherein the materials increasing expression of DUOX 1 gene strengthen skin barrier function.

18. The method for improving function of skin stratum corneum according to claim 14, wherein the materials increasing expression of DUOX 1 gene reduce or treat symptoms of atopy.
